(19) United States
(12) Patent Application Publication
(10) Pub. No.: US 2007/0137643 A1
(43) Pub. Date: Jun. 21, 2007

(54) Fluid dispenser

(76) Inventors: Stanley George Bonney, Hertfordshire (GB); Louise Albert Della-Porta, Warwickshire (GB); Paul Kenneth Rand, Hertfordshire (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/597,689

(22) PCT Filed: Feb. 4, 2005
(86) PCT No.: PCT/GB05/00407

§ 371 Date: Aug. 3, 2006

(30) Foreign Application Priority Data
Feb. 6, 2004 (GB) 0402694.4

(51) Int. Cl. A61M 11/00; B65D 37/00

(52) U.S. Cl. 128/200.23; 222/206

(57) ABSTRACT

A fluid dispenser for dispensing a metered volume of a fluid product having a storage chamber for storing the fluid product in; a dispensing outlet through which the fluid product is dispensable from the dispenser; a metering chamber which is adapted to provide the metered volume of the fluid product for dispensing through the dispensing outlet by movement of the metering chamber between a contracted state and an expanded state, movement of the metering chamber from the contracted state to the expanded state placing the metering and storage chambers in fluid communication to enable the metering chamber to receive from the storage chamber an excess volume of the fluid product comprising the metered volume and a surplus volume; and a bleed arrangement adapted to bleed the surplus volume of the fluid product from the metering chamber is described.

### RELATED APPLICATIONS

This application claims priority from UK patent application No. 0 402 694.4 filed 6 Feb. 2004, the content of which is incorporated herein by reference.

This application is also related to the Applicant's PCT patent applications which have been filed concurrently herewith under the Applicant's references PB60733-A, PB60733-B, PB60733-C, PB60733-D, PB60733-G (all entitled ‘A Fluid Dispenser’) and PB60733-F (entitled ‘A Metering Pump System’) and which respectively claim priority from UK patent application Nos. 0 402 690.2, 0 402 691.0, 0 402 692.8, 0 402 693.6, 0 402 697.7 and 0 402 695.1 all filed 6 Feb. 2004, the contents of all of these applications hereby being incorporated herein by reference.

### FIELD OF THE INVENTION

The present invention relates to a dispenser for dispensing a metered volume of a fluid product and is particularly, but not exclusively, concerned with a dispenser for dispensing a metered volume of a fluid medicament, for instance medicaments having liquid, gaseous, powder or topical (cream, paste etc.) formulations. The invention also has application in the area of consumer healthcare, as in the case of toothpaste, sun cream lotion etc.

### BACKGROUND OF THE INVENTION

Fluid product dispensers having metering mechanisms are known in the art. As an example, in the medical field the use of metered dose inhalers (MDIs) is well established. In a MDI, the fluid product is contained under pressure in a canister having an open end closed off by a valve mechanism. The valve mechanism has a valve body which defines a fixed volume metering chamber through which a valve stem is sealingly slidable between filling and discharging positions. In the filling position, the valve stem places the metering chamber in fluid communication with the canister contents, but isolates the metering chamber from the external environment. Conversely, when the valve stem is moved to the discharge position, the metering chamber is placed in fluid communication with the external environment, but isolated from the canister contents. In this way, a metered volume of fluid product is sequentially transferred to the metering chamber and then discharged to the external environment for inhalation by a patient.

The present invention provides a dispenser for a fluid product having a novel dispensing mechanism.

### SUMMARY OF THE INVENTION

According to an aspect of the present invention there is provided a fluid dispenser according to claim 1 hereof.

Exemplary features of the invention are set out in the other claims hereof and also in the claims of the related applications mentioned above.

Other aspects and exemplary features of the invention are to be found in the exemplary embodiments which will now be described, by way of example only, with reference to the accompanying Figures of drawings.

### BRIEF DESCRIPTION OF DRAWINGS

FIG. 1 is an exploded perspective view of a hand-held, hand-operable intra-nasal fluid dispenser in accordance with the present invention which is configured to operate to dispense a plurality of metered doses of a liquid therefrom, one dose per actuation cycle.

FIGS. 2A to 2I are longitudinal sectional views of the fluid dispenser which sequentially show a complete actuation cycle thereof for dispensing a metered dose of the liquid.

FIG. 3 is a schematic enlargement of area I in FIG. 2F illustrating the opening of an outlet valve of the fluid dispenser during a dispensing mode of operation thereof.

FIG. 4 is a schematic illustration of an alternative container for use in the fluid dispenser which is of the bag-type.

FIGS. 5A to 5G are schematic representations of an alternative valve arrangement for use in the fluid dispenser sequentially showing the movement of inlet and outlet valve control members during the actuation cycle of the fluid dispenser.

### DETAILED DESCRIPTION OF THE EXEMPLARY EMBODIMENTS OF THE INVENTION

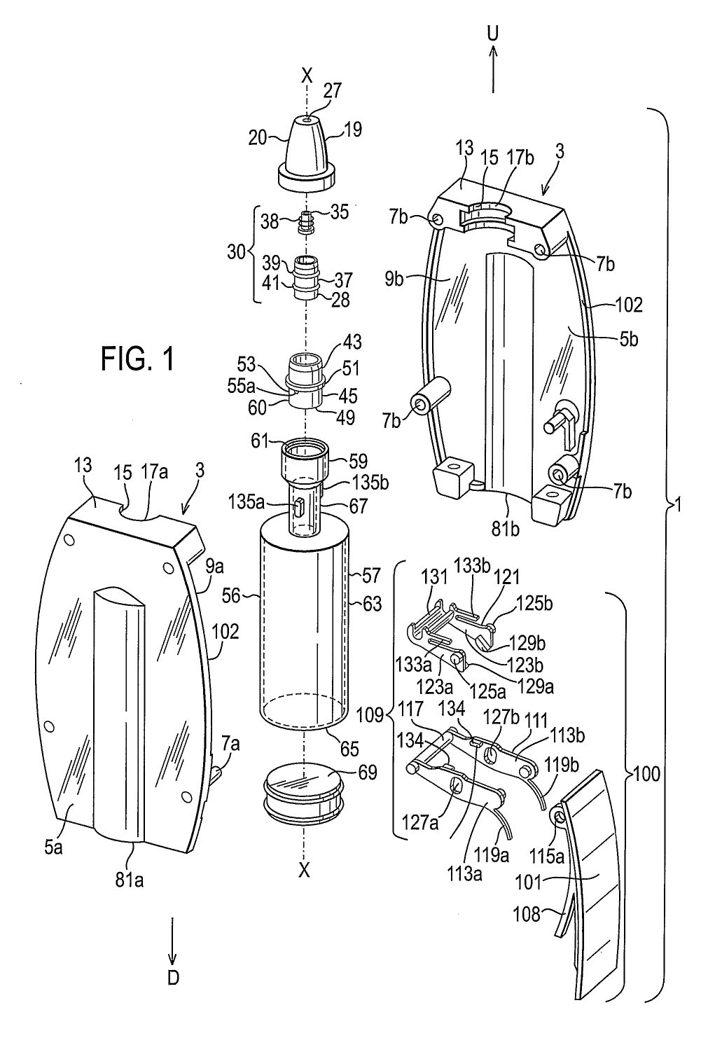

FIGS. 1 to 3 show a fluid dispenser 1 in accordance with the present invention whose underlying principle of operation is as described and claimed in International patent application Nos. PCT/EP03/08646 and PCT/EP03/08647, the entire contents of each of which are hereby incorporated herein by reference.

The fluid dispenser 1 has an outer casing 3 comprising first and second outer casing halves 5a, 5b. The outer casing 3 is assembled through the inter-engagement of complementary male and female connectors 7a, 7b formed on the inner surfaces 9a, 9b of the outer casing halves 5a, 5b. In this particular embodiment, the male connectors 7a are pegs and the female connectors 7b are apertures into which the pegs are slidably receivable.

The outer casing 3 is preferably made from a plastics material, for instance by moulding. Most preferably, the outer casing is made from acrylonitrile-butadiene-styrene (ABS).

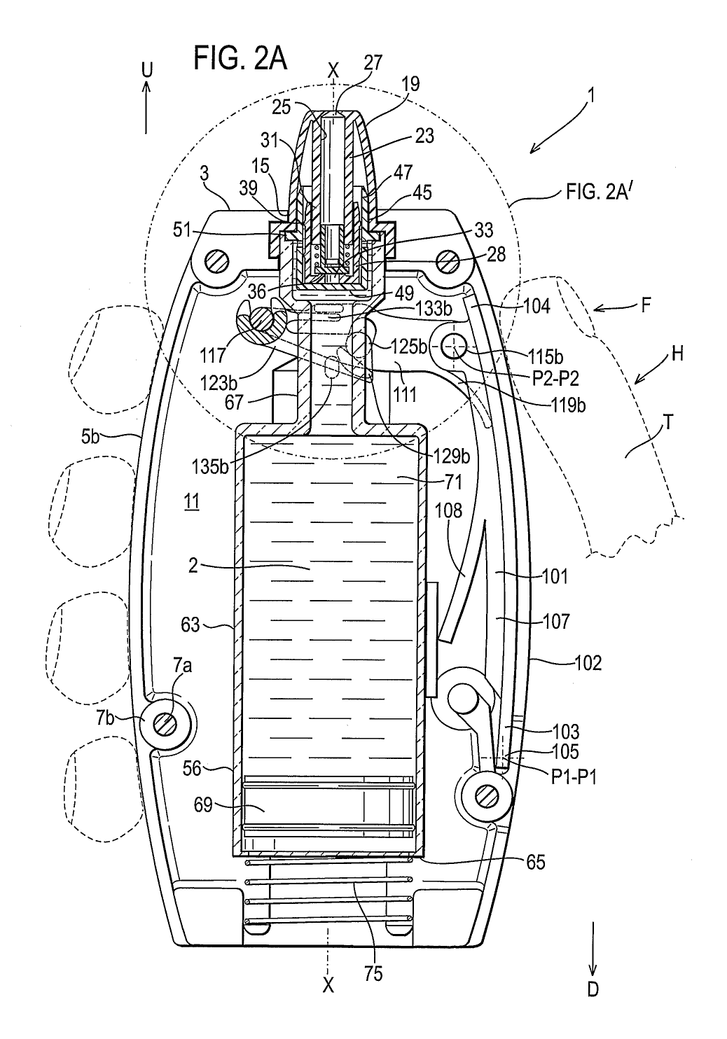

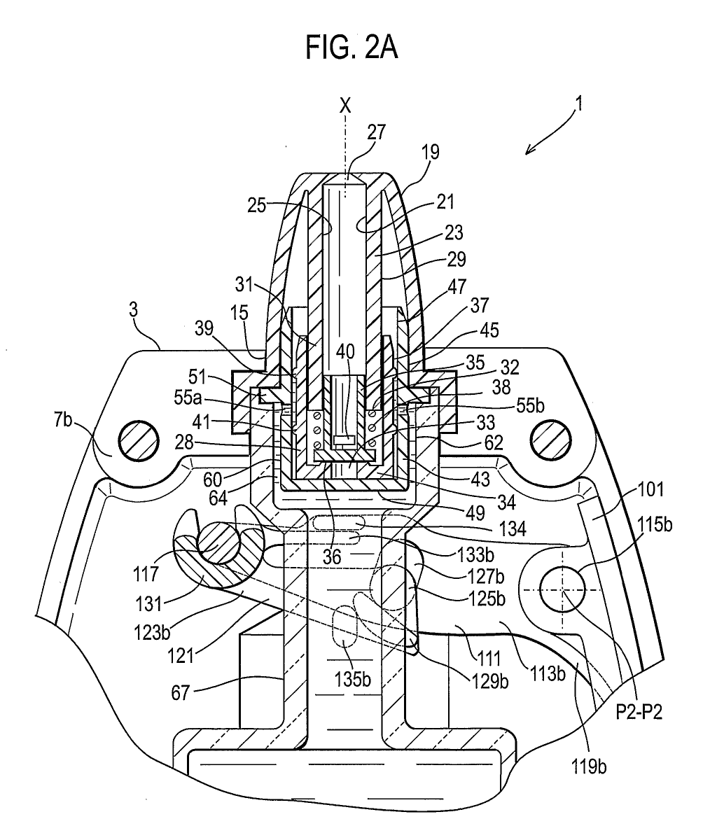

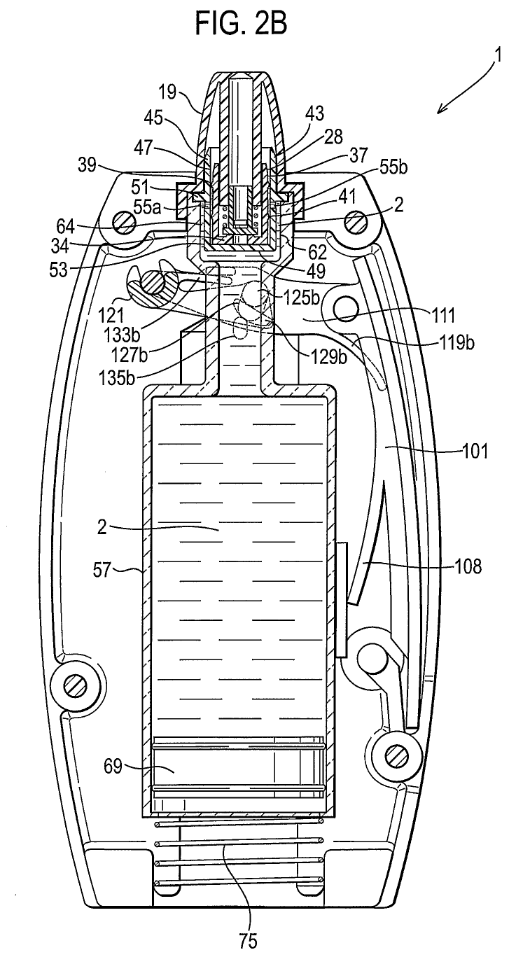

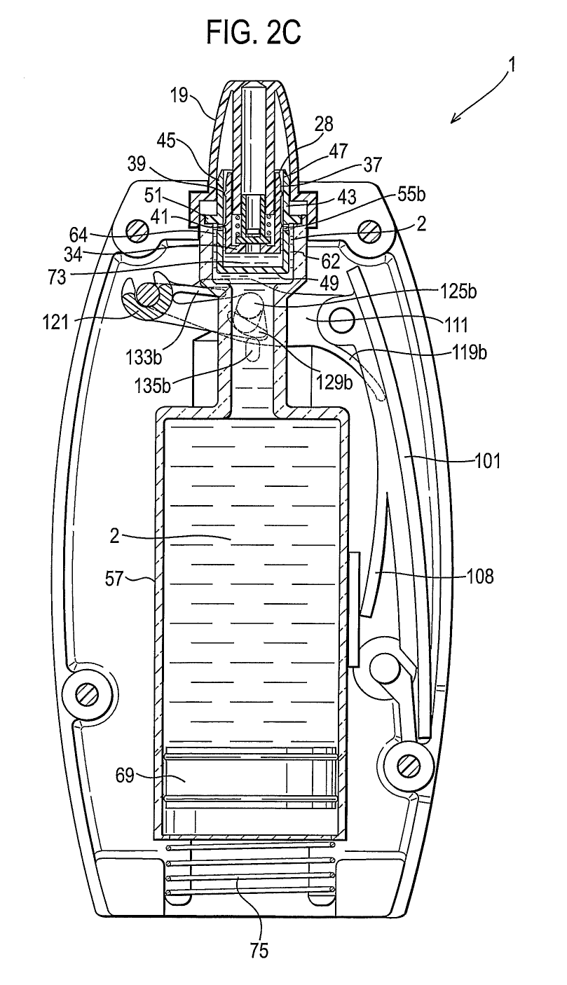

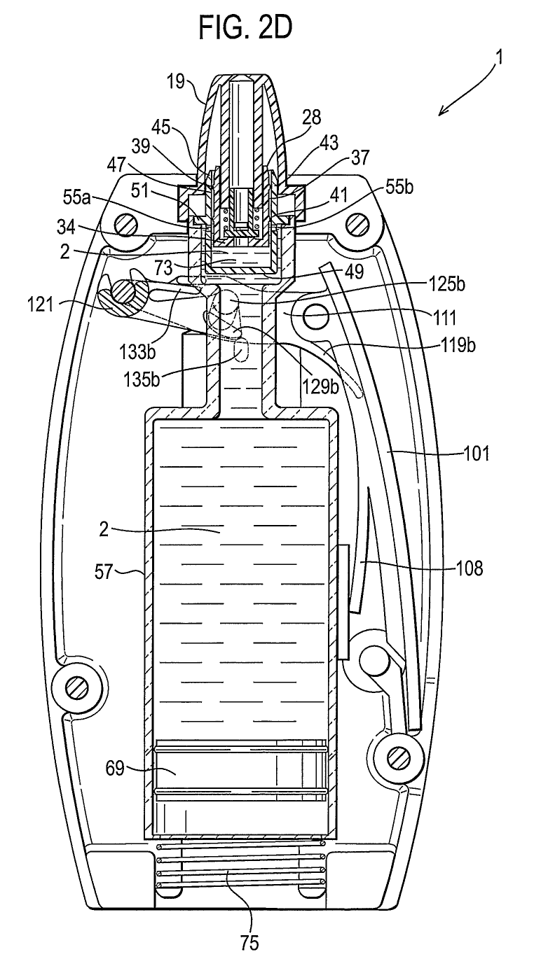

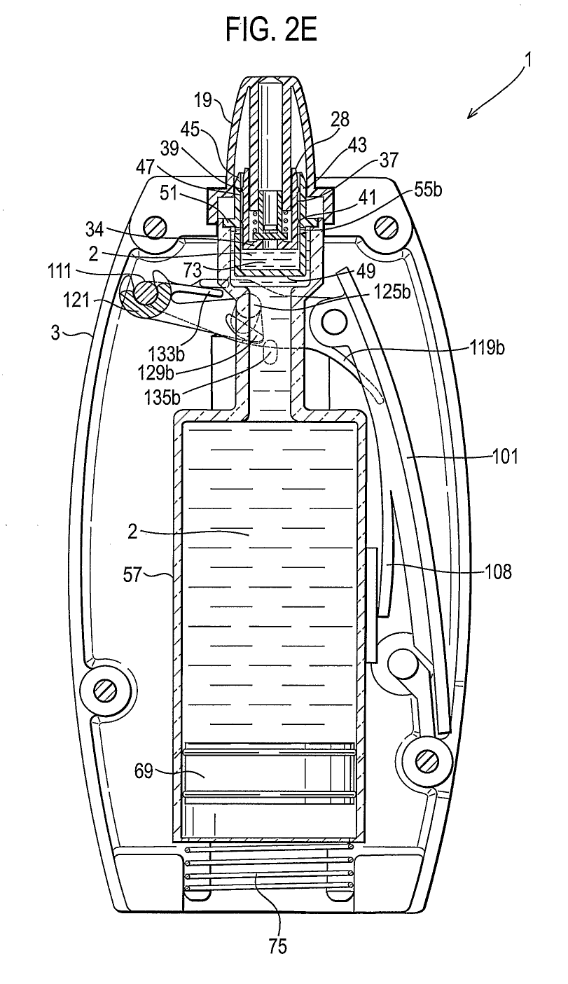

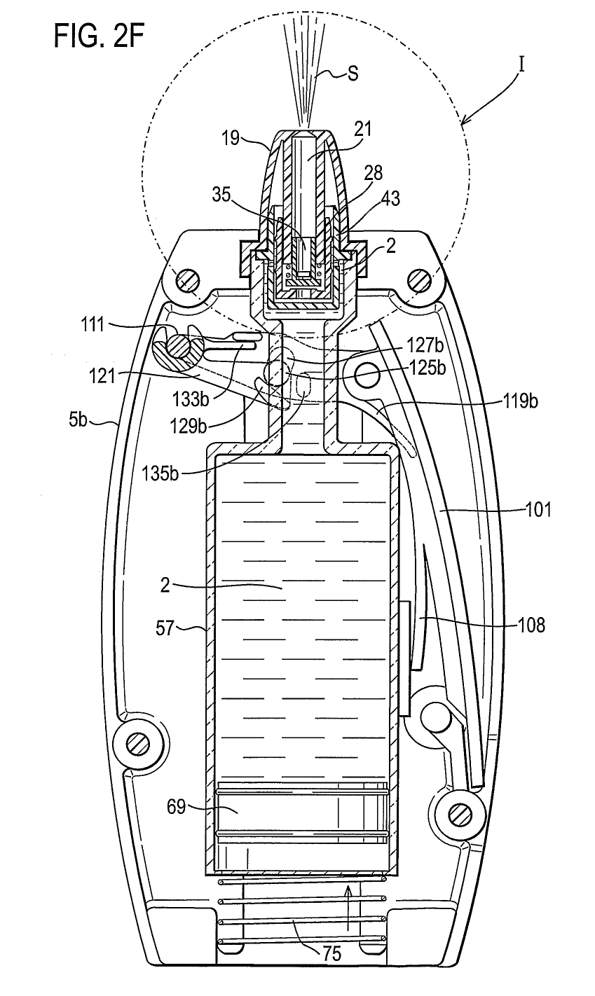

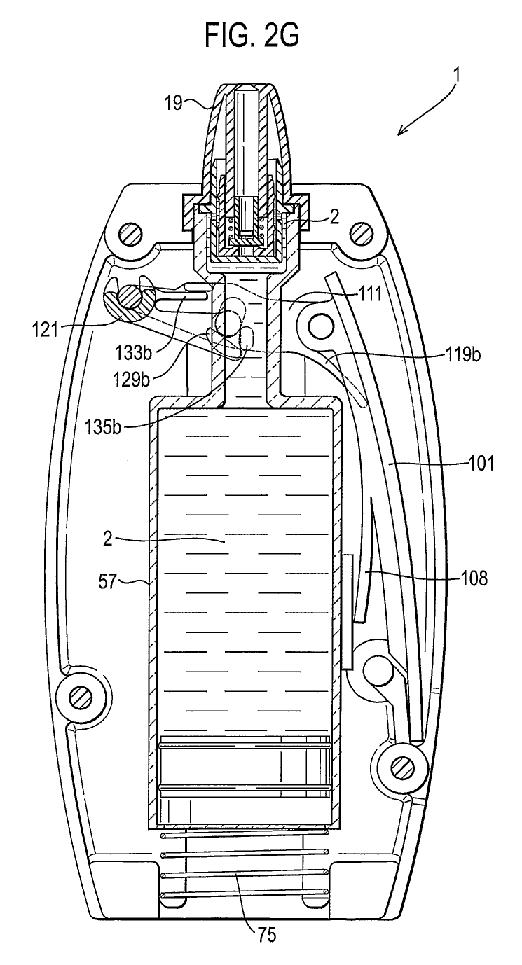

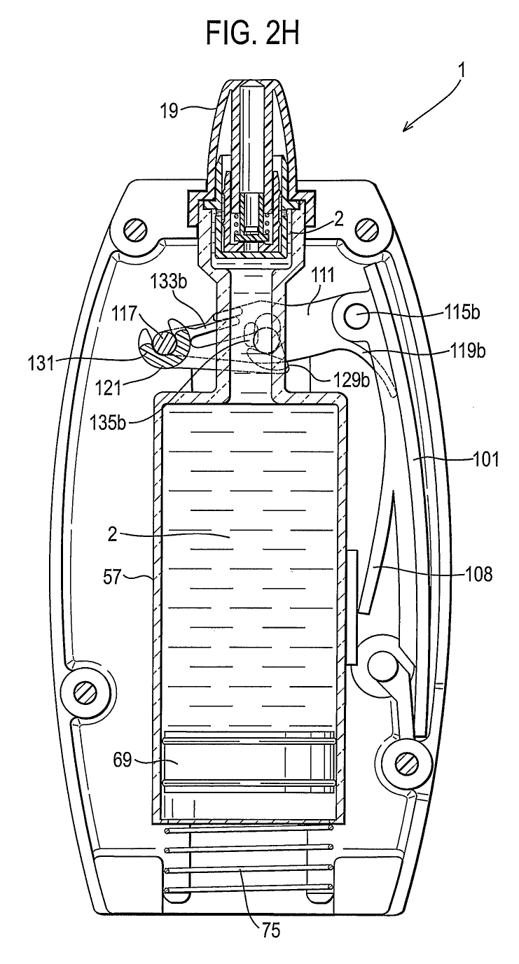

As indicated by the broken line in FIG. 2A, the outer casing 3 of the fluid dispenser 1 is held in the hand H of a human user when operating the fluid dispenser 1. The user's hand H which holds the outer casing 3 is also able to be used to actuate the fluid dispenser 1, as will be understood further hereinafter.

The outer casing halves 5a, 5b have a shell-like form whereby when assembled they enclose an internal chamber 11. As will be understood by reference to FIG. 1, for example, at an upper end 13 of the outer casing 3 there is a passageway 15 to the internal chamber 11 bounded by concave recesses 17a, 17b in the outer casing halves 5a, 5b. The passageway 15 is co-axially arranged with a longitudinal axis X-X of the fluid dispenser 1 and has a generally circular lateral cross section.

The passageway 15 receives a nozzle 19 of the fluid dispenser 1, which in this embodiment is shaped and sized for insertion into a nostril of a human user (i.e. a nasal nozzle). Thus, the fluid dispenser 1 is an intra-nasal fluid dispenser. To this end, the nasal nozzle 19 in this particular embodiment has an outer surface 20 which has a generally circular lateral cross section and which curves laterally inwardly in the upward direction denoted by arrow U.

The nasal nozzle 19 is preferably made from a plastics material, for instance from polypropylene (PP), and may, for example, be formed by moulding.

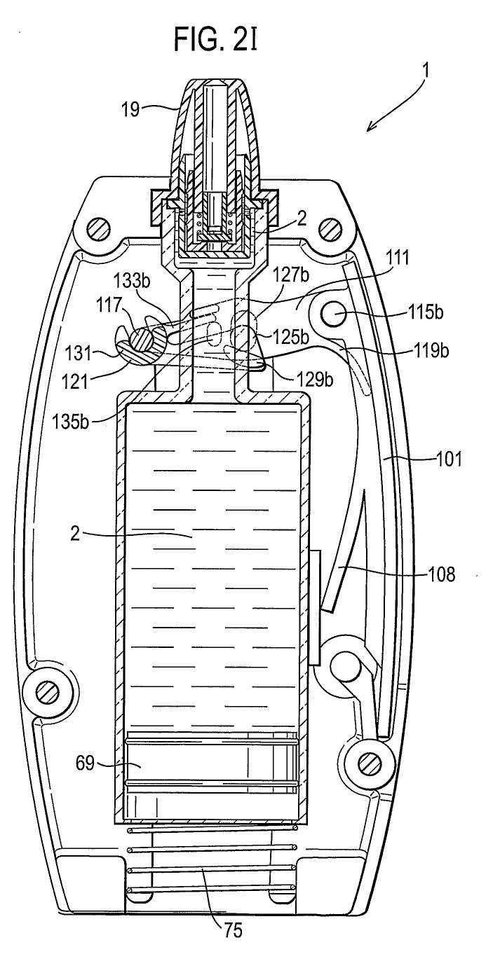

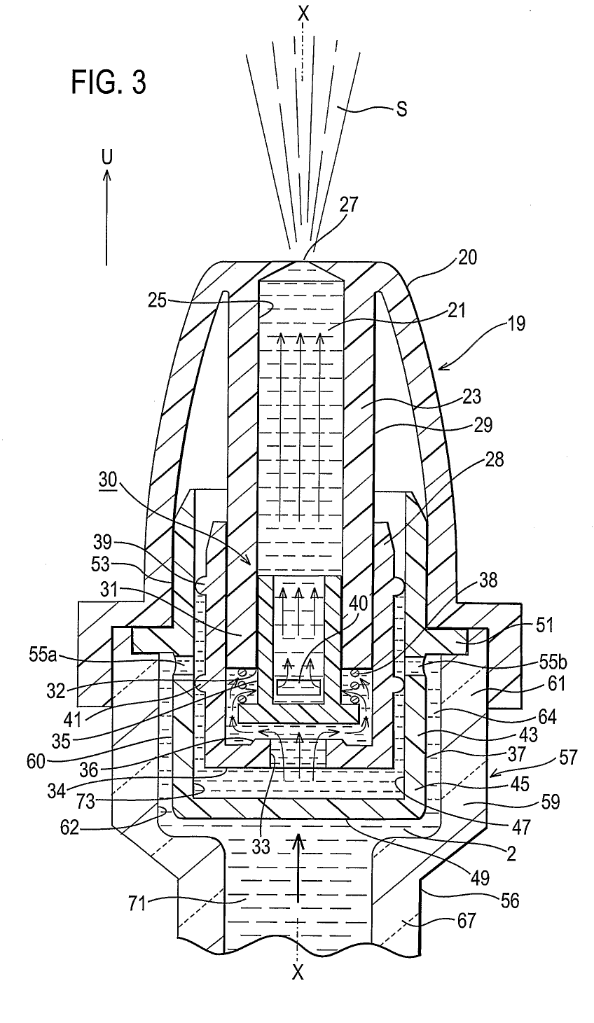

As will be seen from FIGS. 2A and 3, the nasal nozzle 19 is axially aligned with the longitudinal axis X-X and has a longitudinal bore 21 to direct the liquid dispensed from the dispenser 1 in the upward direction U along the longitudinal axis X-X. The nasal nozzle 19 has a generally cylindrical, open-ended inner tubular section 23 whose inner circumferential surface 25 defines the nozzle bore 21. Moreover, the tubular section 23 provides an upper opening 27 of the nozzle bore 21 which is the outlet orifice of the fluid dispenser 1.

As will be appreciated, the nasal nozzle 19 can be of other shapes and configurations suited for insertion into a human nostril.

A generally cylindrical valve body 28 of a one-way (non-return), poppet-type outlet valve 30 is fixedly, sealingly secured on an outer circumferential surface 29 of the nozzle inner tubular section 23 at its lower end 31 so that a lateral lower end wall 34 of the generally U-shaped valve body 28 is disposed underneath a lower opening 32 of the nozzle bore 21. The lateral lower end wall 34 of the valve body 28 includes a valve opening 33 and an outlet valve control member 35 operates in use to selectively place the outlet valve opening 33 and the nozzle bore 21 in flow communication so that a metered volume (metered dose) of the liquid 2 is able to flow through the outlet valve 30 into the nozzle bore 21, as will be described in more detail hereinafter.

The outlet valve control member 35 has a generally cylindrical, tubular stem which is open at its upper end and closed by a flange plate at its lower end. One or more apertures 40 are provided in the tubular stem. The tubular stem is sealingly, slidably mounted in the lower opening 32 of the nozzle bore 21. The outlet valve control member 35 is biased by an outlet valve return spring 38, preferably integrally formed with the outlet valve control member 35, to a rest position in which the flange plate of the outlet valve control member 35 sealingly closes the valve opening 33 by seating on a valve seat 36, as shown in FIG. 2A.

During actuation of the fluid dispenser 1, the outlet valve control member 35 is lifted off the valve seat 36 to place the valve opening 33 in flow communication with the nozzle bore 21 through the one or more apertures 40 in the tubular stem of the outlet valve control member 35, as will be described in more detail hereinafter, particularly with reference to FIG. 3.

The components 28,35 of the metering valve 30 may be made from polypropylene (PP), for example by moulding.

As shown in FIGS. 1 and 3, for example, the valve body 28 has an outer circumferential surface 37 on which is provided upper and lower sealing rings 39, 41. The upper and lower sealing rings 39, 41 may be integrally formed with the valve body 28 or be separate valve components.

As will be observed from a comparison of FIGS. 2A and 2B with FIGS. 2C to 2E, a generally U-shaped sliding member 43 is sealingly, slidably mounted on the outer circumferential surface 37 of the U-shaped valve body 28 for reciprocation along the longitudinal axis X-X between upper and lower positions relative to the U-shaped valve body 28. More particularly, the U-shaped sliding member 43 has a generally circular, longitudinal side wall 45 having an inner circumferential surface 47 which sealingly slides over the upper and lower sealing rings 39, 41 on the valve body 28. The U-shaped sliding member 43 further has a lateral lower end wall 49 which, in the upper position, abuts with the lateral lower end wall 34 of the valve body 28 (see e.g. FIGS. 2A, 2B and 2F to 2I), and which, in the lower position (FIGS. 2D and 2E), is spaced downwardly from the lateral lower end wall 34 of the valve body 28. It can therefore be seen that the U-shaped valve body 28 and the U-shaped sliding member 43 are arranged in a nesting configuration.

The longitudinal side wall 45 of the U-shaped sliding member 43 has an outwardly extending connector flange 51 at an intermediate position of its outer circumferential surface 53. As best illustrated in FIGS. 2B and 3, four equi-angularly spaced transfer ports 55a, 55b (only two shown) extend laterally through the longitudinal side wall 45 of the U-shaped sliding member 43 at a position below the connector flange 51. Of course, the number of transfer ports can be decreased or increased as desired.

In this embodiment, the U-shaped sliding member 43 is made from a plastics material, e.g. by moulding. A preferred plastics material is polypropylene (PP).

A generally cylindrical, liquid-containing hollow container 57 is affixed to the U-shaped sliding member 43 so as to reciprocate therewith on the longitudinal axis X-X. In particular, the container 57 has an open-ended container body 56 having a generally U-shaped head 59 at an upper end 61 which nests with the U-shaped sliding member 43 to be fixedly, sealingly engaged with the connector flange 51 of the U-shaped sliding member 43, e.g. by adherence therebetween. As further best shown in FIGS. 2B and 3, the connection is such that the lower section 60 of the outer circumferential surface 53 of the U-shaped sliding member 43, which is below the connector flange 51, is spaced laterally inwardly of the inner circumferential surface 62 of the U-shaped container head 59 so as to form an annular channel 64 therebetween, which is sealingly closed off at the upper end 61 by the connector flange 51 and into which the transfer ports 55a, 55b open.

The container body 56 further has an enlarged hollow base 63 at a lower end 65 and a hollow neck 67 which extends longitudinally from the base 63 to the head 59. A sealing piston 69 is sealingly, slidably mounted in the container body base 63 to sealingly close the container body 56 at the lower end 65.

In this embodiment the container body 56 is made from glass, although, of course, other inert materials may be used, for example a plastics material, such as polypropylene (PP). Where the container body 56 is made from a plastics material, it can be connected to the flange 51 of the plastics U-shaped sliding member 43 by welding, e.g. by ultrasonic welding.

In this embodiment the sealing piston 69 is made from a plastics material, e.g. by moulding, and is preferably made from butyl rubber.

In this particular embodiment, the container 57 contains a liquid medicament formulation.

As will be appreciated by the skilled reader in the art, the lower end of the annular channel 64 about the U-shaped sliding member 43 is in flow communication with the inner volume of the container body neck 67 which in turn is in flow communication with the inner volume of the closed container body base 63. It will therefore be understood that the container 57 co-operates with the sliding member 43 to define a container inner volume 71 which is only open at the transfer ports 55a, 55b due to the inner volume 71 being sealed by the sealing piston 69 at the lower end 65 and by the connector flange 51 at the upper end 61. For convenience, the assembly of the U-shaped sliding member 43 and the container 57 will now be referred to as the “container unit 58”.

Importantly, as will be appreciated by recourse to FIGS. 2C to 2E and 3, the U-shaped sliding member 43 and the lateral lower end wall 34 of the metering valve body 28 co-operate to define a pumping metering chamber 73 therebetween which is either sealed or selectively open to the transfer ports 55a, 55b or the nozzle bore 21 depending on the sliding position of the container unit 58 on the valve body 28, as will be detailed further hereinafter.

The fluid dispenser 1 is filled with sufficient liquid 2 that, before it is first used, it completely fills the container inner volume 71, including the annular channel 64. Moreover, the fluid dispenser operation is such that the container inner volume 71 is kept airless, i.e. there is no headspace.

As shown in FIG. 2A, for example, a return spring 75 of compression type acts on the container base 63 to bias the container unit 58 in the upward direction U to an upper sliding position in the outer casing 3 in which the U-shaped sliding member 43 is disposed in its upper position on the valve body 28. As will be understood more fully shortly hereinafter, the fluid dispenser 1 is adapted so that, in its rest or non-actuated state, the container unit 58 is placed in the upper sliding position by the return spring 75.

As illustrated in FIGS. 2A and 2B, for example, the upper sliding position of the container unit 58 is defined by the abutment of the lateral lower end wall 49 of the U-shaped sliding member 43 with the lateral lower end wall 34 of the valve body 28 (i.e. when the U-shaped sliding member 43 is in its upper sliding position on the valve body 28). It will thus be appreciated that the pumping metering chamber 73 has no, or substantially no, volume in the rest state of the fluid dispenser 1. Moreover, in the upper sliding position of the U-shaped member 43 the transfer ports 55a, 55b are disposed in-between the upper and lower sealing rings 39, 41 on the valve body 28. Furthermore, the outlet valve control member 35 is in its closed position. Consequently, the metering chamber 73 is not in flow communication with the inner volume counter 71 of the container 57 nor with the nozzle bore 21. That is to say, the metering chamber 73 is sealed.

Thus, the inner volume 71 of the container unit 58 is completely sealed in the rest state of the fluid dispenser 1 inasmuch as contaminants, such as air and moisture, cannot enter the container inner volume 71 at its lower end 65, due to the sealing piston 69, nor at the upper end 61 by virtue of the position of the transfer ports 55a, 55b between the sealing rings 39, 41, the collapsed state of the metering chamber 73 and the closed position of the outlet valve control member 35. Of course, it will be appreciated that the components of the fluid dispenser 1 are made from fluid impervious materials.

As will be described in more detail shortly hereinafter, the fluid dispenser 1 is provided with a hand-operable actuating mechanism 100 for reciprocating the container unit 58 along the longitudinal axis X-X to cause a metered dose of the liquid 2 to be dispensed.

In broad terms, the actuating mechanism 100 drives the container unit 58 downwardly in the direction of arrow D against the return force of the return spring 75. In so doing, the U-shaped sliding member 43 parts from the valve body 28 so as to increase the volume of the metering chamber 73, as shown in FIGS. 2C to 2E. This results in a negative pressure or vacuum being produced in the metering chamber 73. Eventually, the transfer ports 55a, 55b slide past the lower sealing ring 41 to place the metering chamber 73 and the container inner volume 71 in flow communication with one another. Liquid from the container 57 is then drawn into the metering chamber 73 due to the negative pressure created in the metering chamber 73 during the downward stroke of the container unit 58. In this regard, the sealing piston 69 slides up in the container base 63, under the influence of the negative pressure, to decrease the inner volume 71 of the container 57 by an amount equivalent to the liquid volume transferred into the metering chamber 73. Accordingly, no headspace is generated over the liquid 2 in the container 57 during the filling of the metering chamber 73.

It is to be noted that the outlet valve control member 35 remains closed in the downward stroke to prevent escape of any of the liquid 2 transferred into the metering chamber 73 during this filling mode of operation of the fluid dispenser 1.

Once the downward stroke is completed, and the container unit 58 is at its lower sliding position shown in FIG. 2E, the return spring 75 is released to drive the container unit 58 upwards and to compress the metering chamber 73. To this end, the hydraulic force needed to cause the sealing piston 69 in the container base 63 to slide downwards is less than that required to open the outlet valve control member 35. As a result, during an initial phase of the upward return stroke of the container unit 58 in the outer casing 3 a proportion of the liquid 2 in the metering chamber 73 is bled back to the container inner volume 71 via the transfer ports 55a, 55b resulting in the sealing piston 69 sliding downwardly in the container base 63. This is the bleed mode of operation of the fluid dispenser 1.

In the bleed mode of operation the sealing piston 69 moves downwardly to a new rest position which is spaced upwardly of its previous rest position before the filling mode of operation. The increase in the container inner volume 71 in the bleed mode is equivalent to the volume of liquid bled back thereinto. Thus, no headspace is created in the container inner volume 71 in the bleed mode.

At an intermediate sliding position of the container unit 58 during the upward return stroke, not shown, the transfer ports 55a, 55b are juxtaposed with the lower sealing ring 41 so as to be closed thereby. At this point in the upward return stroke no more liquid 2 is able to be bled back to the container 57. Moreover, the metering chamber 73 now defines the metering volume of the fluid dispenser 1 and is filled with a metered volume of the liquid 2 transferred thereinto during the filling mode of operation. In this particular embodiment, the metering volume is 50 μL, although, of course, the fluid dispenser 1 can be made to produce other metering volumes depending on the specific application and/or product to be dispensed.

During the final phase of the upward return stroke of the container unit 58, in which the container unit 58 slides from the intermediate sliding position to the upper sliding position, the volume of the metering chamber 73 continues to reduce to increase the hydraulic pressure therein causing the outlet valve control member 35 to lift off the outlet valve seat 36 and the metered volume of liquid 2 to be pumped from the metering chamber 73 out of the dispenser outlet orifice 27 via the nozzle bore 21. This is the dispensing mode of operation of the fluid dispenser 1 and is shown schematically in FIG. 3. At the end of the return stroke the outlet valve control member 35 re-closes the outlet valve opening 33.

As will be appreciated, an actuation cycle of the fluid dispenser 1 results in the sealing piston 69 moving upwardly by an amount which results in the container inner volume 71 reducing by the metered volume. This ensures that no headspace is provided in the container inner volume 71 thereby ensuring no air is present therein. Accordingly, repeated use of the fluid dispenser 1 causes the sealing piston 69 to move incrementally upwardly until it bears against the roof 66 of the container base 63 whereupon no further dispensing takes place.

The use of the return spring 75 to drive the container unit 58 upwardly for the bleed and dispensing modes removes human force inconsistencies from the use of the fluid dispenser 1.

The pumping force of the fluid dispenser 1 is such as to produce an atomised spray having a relative small and uniform droplet size ideal for delivery to the nasal passage of the user. For example, the fluid dispenser 1 may be adapted to dispense the metered volume as a spray of droplets having a diameter in the range of 10-20 μm.

Mindful of the above description of the pumping action produced by reciprocation of the container unit 58 in the outer casing 3 along the longitudinal axis X-X, it will be seen that actuation of the actuation mechanism 100 of the fluid dispenser 1 has three sequential effects, namely:
(1) Creating a filling mode in which an excess volume of the liquid 2 is drawn from the container 57 into the metering chamber 73 by the negative pressure created in the metering chamber 73 as it expands.
(2) Creating a bleed mode in which the surplus volume of the liquid 2 in the metering chamber 73 is bled back to the container 57 to leave a metered volume in the metering chamber 73 as the metering chamber 73 begins to be compressed.
(3) A dispensing mode in which the metered volume is pumped from the dispenser 1 as the metering chamber 73 completes its compression to zero, or substantially zero, volume.

Each further actuation of the actuating mechanism 100 results in this cycle of events being repeated until the sealing piston 69 abuts the roof 66 of the container base 63. In this particular embodiment, the inner volume 71 of the container base 63, which corresponds to the volume of liquid 2 that is dispensable from the fluid dispenser 1, is 14 ml. Consequently, the fluid dispenser 1 has 280 actuations.

By way of example, the container 57 can be filled with the liquid 2 after it has been assembled into the fluid dispenser 1 by forming the sealing piston 69 so that it is able to be sealingly pierced by a needle-like object and then sealably reclose after withdrawal of the needle-like object (e.g. a “septum”). In this way, the liquid could be injected through the sealing piston 69. To this end, it will noted from FIG. 1 that the outer casing halves 5a, 5b each have a base with a concave cut-out 81a, 81b which, when the outer casing 3 is assembled, provide an aperture in the outer casing base. The injector could be inserted through the sealing piston 69 via this aperture.

An alternative filling method is vacuum filling, as will be understood by the skilled person in the art.

A description of the actuation mechanism 100 will now be given with reference to FIGS. 2 and 3. The actuation mechanism is lever-based in the sense that actuation is effected through an actuation lever 101 which is mounted to the outer casing 3 in a longitudinal slot 102 thereof formed by the junction of opposed sides of the outer casing halves 5a, 5b.

The actuation lever 101 has a lower end 103 which is pivotally connected to the outer casing 3 at a pivot point 105 for pivotal movement about a first lateral pivot axis P1-P1. The actuation lever 101 has an inner surface 107 from which depends a return leaf spring 108. The return leaf spring 108, which is preferably an integrally formed part of the lever 101, co-operates with the container base 63 to bias the actuation lever 101 to an outward rest position in which it forms a flush fit in the outer casing 3, as shown in FIG. 2A, for example. This is the position the actuation lever 101 adopts in the non-actuated or rest state of the fluid dispenser 1.

As illustrated in FIGS. 2A to 2C, to actuate the actuating mechanism 100 the user picks up the fluid dispenser 1 in their hand H and pushes the actuation lever 101 from its outward rest position into the outer casing 3 to cause it to pivot about the first pivot axis P1-P1 against the return force of the leaf spring 108. The user uses a digit of the hand H holding the fluid dispenser 1 to push the actuation lever 101 inwardly, in this instance their thumb T. The actuation lever 101 is returned to the outward return position upon release, or relaxation, of the pushing force F on the actuation lever 101 by the return spring 108.

In this particular embodiment, the user pushes the actuation lever 101 inwardly after the nozzle 19 has been inserted into one of their nostrils.

Mounted to the inner surface 107 of the actuation lever 101 at an upper end 104 thereof is a laterally extending drive structure 109 which is so constructed and arranged in the fluid dispenser 1 to transmit the inward pivotal motion of the actuation lever 101 into a downward driving force on the container unit 58 to effect the downward stroke thereof, as described hereinabove.

More particularly, the drive structure 109 has a generally U-shaped outer carrier frame 111 pivotally connected to the actuation lever 101 for pivotal movement about a second lateral pivot axis P2-P2 which extends generally parallel to the first pivot axis P1-P1. The U-shaped outer carrier frame 111 has a pair of generally parallel side members 113a, 113b which straddle the neck 67 of the container 57 on opposed sides thereof and are connected at first ends thereof to pivot points 115a, 115b on the actuation lever inner surface 107, and a crossbar member 117 which connects the side members 113a, 113b at second ends thereof. Thus, the U-shaped outer carrier frame 111 forms a hollow box-like structure with the actuation lever 101 which encloses the neck 67 of the container 57.

The U-shaped outer carrier frame 111 further has a return leaf spring 119a, 119b depending from the first end of each side member 113a, 113b which co-operates with the inner surface 107 of the actuation lever 101 to bias the U-shaped carrier frame 111 to an upper pivot position which, for example, is shown in FIG. 2A.

The drive structure 109 further comprises a generally U-shaped inner cam frame 121 which is carried by the U-shaped outer carrier frame 111 on the inside thereof. The inner cam frame 121 has a pair of generally parallel side members 123a, 123b which are arranged generally parallel to the side members 113a, 113b of the outer carrier frame 111. The inner cam frame side members 123a, 123b are each provided with an outwardly projecting lug 125a, 125b at a first end thereof which is received in a longitudinal slide aperture 127a, 127b formed in the adjacent outer carrier frame side member 113a, 113b between the first and second ends thereof.

The inner cam frame side members 123a, 123b are also each provided with an inwardly projecting cam element 129a, 129b of wing-like cross-section, the function of which will be outlined further hereinafter.

The inner cam frame 121 further has a crossbar member 131 which connects the side members 123a, 123b at second ends thereof. The inner cam frame crossbar member 131 is configured as a C-shape clip which clips to the crossbar member 117 of the outer carrier frame 111 to enable the inner cam frame 121 to be pivotal thereabout.

The pivotal movement of the inner cam frame 121 on the outer carrier frame 111 is governed by sliding movement of the lugs 125a, 125b in the associated slide apertures 127a, 127b. Specifically, the end limits of the pivotal movement of the inner cam frame 121 about the crossbar member 117 of the outer carrier frame 111 between lower and upper pivot positions are respectively determined by the abutment of the lugs 125a, 125b with the lower and upper ends of the longitudinal slide apertures 127a, 127b.

In this regard, and referring to FIG. 1, the inner cam frame 121 yet further comprises a return leaf spring 133a, 133b projecting upwardly from each opposing end of the crossbar member 131. The return leaf springs 133a, 133b of the inner cam frame 121 each co-operate with an abutment surface 134 on the adjacent outer carrier frame side member 113a, 113b to bias the inner cam frame 121 in the downward direction D to its lower pivot position. Thus, in the rest state of the fluid dispenser 1 shown in FIG. 2A, for example, the lugs 125a, 125b of the inner cam frame 121 are held against the lower ends of the slide apertures 127a, 127b of the outer carrier frame 111.

The function of the inner cam frame 121 is to convert the inward movement of the actuation lever 101 into a downward camming action on the container unit 58 and thereby place the fluid dispenser 1 in its filling mode. To this end, a pair of diametrically opposed peg-shaped cam followers 135a, 135b (only one shown) extend laterally from the neck 67 of the container 57. The cam followers 135a, 135b and cam elements 129a, 129b on the inner cam frame 121 co-operate to produce the downward stroke of the container unit 58 representing the filling mode, as will now be described in more detail.

When the fluid dispenser 1 is in its rest state, the component parts thereof adopt the relative positions shown in FIG. 2A. Notably, the container unit 58 is held in its upper slide position by the return spring 75, the actuation lever 101 is in its outward pivot position, the outer carrier frame 111 is in its upper pivot position and the inner cam frame 121 is in its lower pivot position.

Referring to FIGS. 2A and 2B, to actuate the actuation mechanism 100 the actuation lever 101 is pivoted inwardly, as discussed previously, and this pivotal inward movement is transmitted to the drive structure 109 causing it to be displaced laterally inwardly. In an initial phase of the inward movement of the drive structure 109, the inner carrier frame 121 is moved from its lower pivot position relative to the outer carrier frame 111 to its upper pivot position as a result of the cam elements 129a, 129b riding up the upper surfaces of the cam followers 135a, 135b. In other words, the lugs 125a, 125b are caused to slide upwardly in the slide apertures 127a, 127b from the lower end of the slide apertures 127a, 127b to the upper ends with concomitant compression of the inner cam frame leaf springs 133a, 133b.

Once the lugs 125a, 125b reach the upper ends of the slide apertures 127a, 127b, the inner carrier frame 121 is “locked” in its upper pivot position.

Referring to FIGS. 2C and 2D, continued inward movement of the actuation lever 101 leads to an intermediate phase of inward movement of the drive structure 109 in which the cam elements 129a, 129b act on the cam followers 135a, 135b to displace the container unit 58 in the downward direction D to its lower slide position against the return force of the return spring 75. This moves the fluid dispenser 1 into its filling mode in which the metering chamber 73 is expanded and placed in flow communication with the liquid 2 in the container 57.

Referring to FIGS. 2E and 2F, further continued inward movement of the actuation lever 101 leads to a terminal phase of inward movement of the drive structure 109 in which the cam elements 129a, 129b disengage from the cam followers 135a, 135b whereby the return spring 75 operates to return the container unit 58 to its upper slide position. This moves the fluid dispenser 1 sequentially through its bleed and dispensing modes of operation described hereinabove so that a metered volume of the liquid 2 is discharged from the nasal nozzle 19 as an atomised spray S (FIGS. 2F and 3) into the user's nasal cavity. FIG. 3 shows in detail how the outlet valve control member 35 is lifted off the outlet valve seat 36 during the dispensing mode by the hydraulic pressure built up in the metering chamber 73 once the metering chamber 73 is sealed after the bleed mode. As indicated by the arrows, this allows the liquid 2 to be pumped through the outlet valve aperture 33, around the side of the outlet valve control member 35, through the aperture(s) 40 in the outlet valve control member 35 and out of the outlet orifice 27 via the nozzle bore 21.

Furthermore, once the cam elements 129a, 129b disengage from the cam followers 135a, 135b the return leaf springs 133a, 133b of the inner cam frame 121 are free to slide the lugs 125a, 125b downwardly in the slide apertures 127a, 127b to return the inner cam frame 121 to its lower slide position on the outer carrier frame 111. This is shown most clearly in FIG. 2F.

As shown in FIG. 2E, for instance, the inward movement of the drive structure 109 is delimited by abutment of the crossbar 131 of the inner cam frame 121 with an inner surface of the outer casing 3.

Once the fluid dispenser 1 has dispensed the metered volume of liquid, the user can remove or reduce the inward displacement force F on the actuation lever 101 to allow the actuation lever return leaf spring 108 to return the actuation lever 101 to its outward rest position to reset the fluid dispenser 1 in its rest mode in preparation for its next use. This sequence is shown in FIGS. 2G to 2I from which it will be noted that, in an initial phase of the concomitant returning outward movement of the drive structure 109, the cam elements 129a, 129b re-engage the cam followers 135a, 135b, albeit this time riding over the lower cam follower surfaces due to the lugs 125a, 125b now being at the lower ends of the slide apertures 127a, 127b. Moreover, for the same reason, the outer carrier frame 111 tilts to its lower pivot position on the actuation lever 101.

Towards the end of the return movement of the actuation mechanism 100 to its rest state, the cam elements 129a, 129b disengage from the cam followers 135a, 135b thereby enabling the outer carrier frame 111 and inner cam frame 121 to return to their respective rest states.

In this embodiment, the actuation lever 101, the outer carrier frame 111 and the inner cam frame 121 are made from a plastics material, for instance ABS, as an example by moulding.

In a modification of the fluid dispenser 1, the container 57 may be replaced by a bag structure which would contract and expand in equivalent fashion, and for equivalent function, as the container 57, e.g. by being made from a flexible material, for instance a plastics material. An advantage of a bag structure over the container 57 would be that it avoids the need for a complex structure for contraction and expansion of its inner volume.

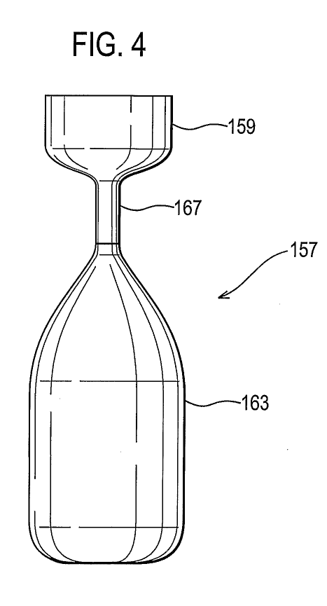

An example of a bag container 157 is shown in FIG. 4 with like reference numerals indicating like features in the container 57 of FIGS. 1 to 3. The bag container 157 has a head 159 and a neck 167 corresponding to those in the container 57. The base 163 of the bag container 157 is formed by a bag element which expands/contracts depending on the mode of operation of the fluid dispenser 1.

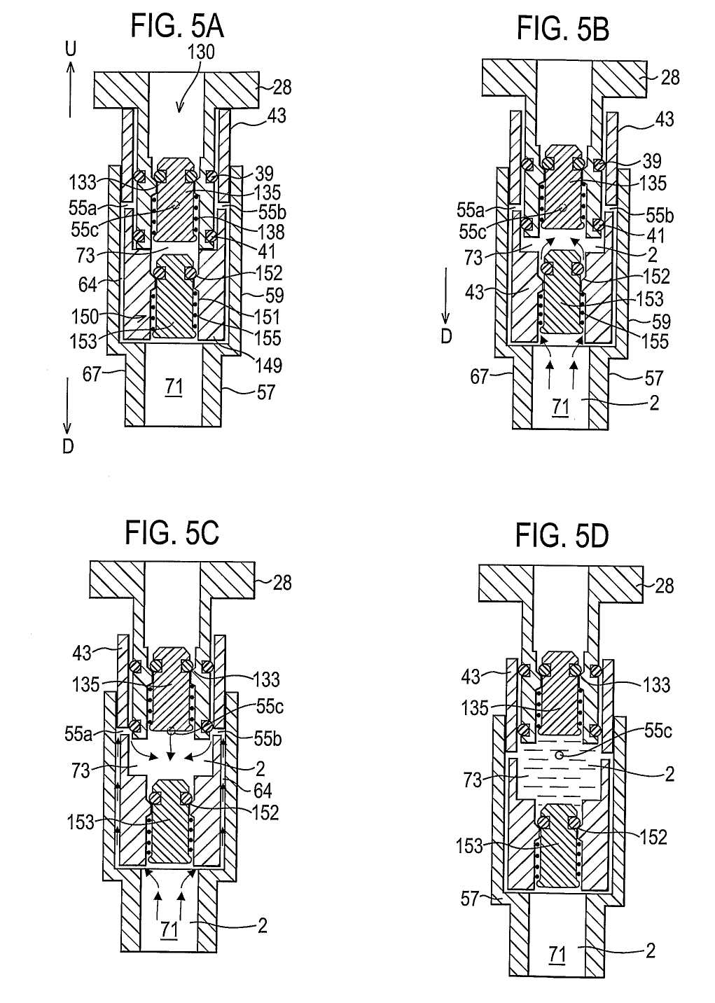

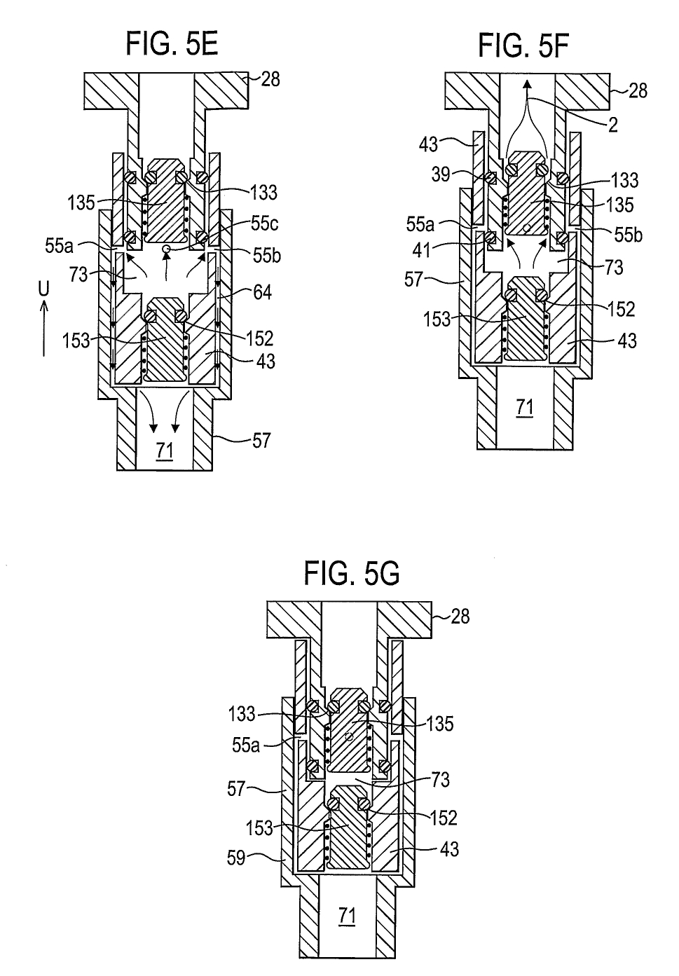

Referring now to FIGS. 5A to 5G, there is shown an alternative valve arrangement for use in the fluid dispenser 1 of FIGS. 1 to 3. For simplicity, those features in the alternative valve arrangement which are equivalent to features of the valve arrangement shown in FIGS. 1 to 3 are ascribed like reference numerals.

As shown in FIGS. 5A to 5G, a relief inlet valve 150 is positioned between the metering chamber 73 and the inner volume 71 of the container 57 which remains closed other than when the downstroke of the container unit 58 is initiated whereupon it is temporarily caused to open by the reduced pressure created in the metering chamber 73 during this phase. This allows liquid 2 to enter the metering chamber 73 before the transfer ports 55a-c (three shown this time) are placed in flow communication with the metering chamber 73. This makes it easier to move the container unit 58 in the downward direction D against the reduced pressure in the metering chamber 73 until the transfer ports 55a-c are opened, whereupon liquid 2 enters the metering chamber 73 therethrough. This results in the pressure in the metering chamber 73 increasing which biases the inlet valve 150 back to its shut position. Filling of the metering chamber 73 then continues through the transfer ports 55a-c as previously described with reference to FIGS. 1 to 3.

More particularly, the inlet valve 150 has an inlet valve opening 151 in the lateral lower end wall 49 of the U-shaped sliding member 43 and an inlet valve control element 153 slidably, sealingly mounted in the inlet valve opening 151 for movement between a closed position, shown in FIG. 5A, in which the inlet valve control element 153 is seated on an inlet valve seat 152 to shut the inlet valve opening 151 to prevent flow communication between the metering chamber 73 and the inner volume 71 of the container 57, and an open position, shown in FIG. 5B, in which the inlet valve control element 153 moves off the inlet valve seat 152 to open the inlet valve opening 151 to put the metering chamber 73 and the inner volume 71 of the container 57 in flow communication. The inlet valve 150 further has a return spring 155 which biases the inlet valve control element 153 to its closed position.

FIG. 5A shows that the inlet valve control element 153 is biased by the return spring 155 to the closed position in the rest state of the fluid dispenser 1. When the actuation mechanism 100 is actuated by inward displacement of the actuation lever 101, the U-shaped sliding member 43 is moved downwardly with respect to the outlet valve body 28 causing the metering chamber 73 to expand from its contracted state. The reduced or negative pressure this creates in the metering chamber 73 draws the inlet valve control element 153 up off the inlet valve seat 152 to its open position against the return force of the inlet valve return spring 155. The reduced pressure in the metering chamber 73 then draws liquid 2 into the metering chamber 73 from the container 57 through the inlet valve opening 151, as shown in FIG. 5B. At this point the transfer ports 55a-c are still shut in the sense that they have not travelled below the lower sealing ring 41.

As the downward movement of the U-shaped sliding member 43 continues during the filling mode of operation of the fluid dispenser 1, the metering chamber 73 continues to expand and draw in liquid 2 through the inlet valve 150 until the transfer ports 55a-c open so liquid 2 can be drawn into the metering chamber 73 through these, as shown in FIG. 5C. As further shown by FIG. 5C, as the pressure in the metering chamber 73 increases on intake of liquid 2 thereinto, the return force of the inlet valve return spring 155 biases the inlet valve control element 153 back onto the inlet valve seat 152 to close the inlet valve aperture 151.

The metering chamber 73 is then filled up through the transfer ports 55a-c as the U-shaped sliding member 43 completes its downward stroke. As shown in FIGS. 5A to 5D, the outlet valve 130 remains shut during the whole of the downward stroke. Specifically, the outlet valve control element 135 is biased by the outlet valve return spring 138 into sealing engagement in the outlet valve aperture 133 (the closed position).

FIGS. 5E to 5G depict the upward stroke of the container 57 from which it will be seen that the inlet valve 150 stays shut. FIGS. 5F and 5G show that after the transfer ports 55a-c are re-closed by the lower sealing ring 41, the hydraulic pressure in the metering chamber 73 is sufficient to open the outlet valve 130 to enable discharge of the metered volume contained in the metering chamber 73. Specifically, as shown in FIG. 5F, the hydraulic pressure created in the metering chamber 73 forces the outlet valve control element 135 to slide upwardly in the outlet valve aperture 133 against the biasing force of the outlet valve return spring 138 to enable the liquid in the metering chamber 73 to pass through the outlet valve 130 to the outlet orifice 27 (the open position). As shown in FIG. 5G, once the metered volume has been dispensed, the outlet valve return spring 138 returns the outlet valve control element 135 to its closed position.

The outlet and inlet valve control members 135, 153 may be made from a plastics material, such as polypropylene (PP), for example by moulding.

The fluid dispenser 1 described above provides for high accuracy dosing from a sealed system which protects the liquid 2 from contamination from the external environment. For instance, the non-return outlet valve 30; 130 prevents air ingress. Moreover, the container inner volume 71 is isolated from the outlet orifice 27 by the outlet valve 30; 130 and the closure of the outlet valve aperture 33 by the U-shaped sliding member 43 in the rest state of the dispenser. Accordingly, the liquid can be preservative-free, of particular benefit when the liquid is a medicament.

The dispenser 1 further dispenses without the need for a dip tube, and there is no drain back.

Other advantages of the fluid dispenser 1 that may be mentioned are, without limitation:
Its compactness due to its in-line arrangement, as compared, for example, with the dispenser disclosed in International patent application Nos. PCT/EP03/08646 and PCT/EP03/08647.
The need for the user to only move the actuating lever 101 in a single direction to produce a complete actuation cycle.

Where the dispenser of the invention is a medicament dispenser, for instance an intra-nasal medicament dispenser, administration of the medicament may be indicated for the treatment of mild, moderate or severe acute or chronic symptoms or for prophylactic treatment.

Appropriate medicaments may thus be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone (e.g. as the furoate ester), ciclesonide, triamcinolone (e.g. as the acetonide), 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester or 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; PDE4 inhibitors e.g. cilomilast or roflumilast; leukotriene antagonists e.g. montelukast, pranlukast and zafirlukast; [adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate)]; [α4 integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy) acetyl]amino}pentanoyl)amino] propanoic acid (e.g. as free acid or potassium salt)], diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagons. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament and/or to minimise the solubility of the medicament in the propellant.

Preferably, the medicament is an anti-inflammatory compound for the treatment of inflammatory disorders or diseases such as asthma and rhinitis.

The medicament may be a glucocorticoid compound, which has anti-inflammatory properties. One suitable glucocorticoid compound has the chemical name: 6α, 9α-Difluoro-17α-(1-oxopropoxy)-11β-hydroxy-16α-methyl-3-oxo-androsta-1,4-diene-17β-carbothioic acid S-fluoromethyl ester (fluticasone propionate). Another suitable glucocorticoid compound has the chemical name: 6α, 9α-difluoro-17α-[(2-furanylcarbonyl)oxy]-11β-hydroxy-16α-methyl-3-oxo-androsta-1,4-diene-17β-carbothioic acid S-fluoromethyl ester. A further suitable glucocorticoid compound has the chemical name: 6α,9α-Difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester.

Other suitable anti-inflammatory compounds include NSAIDs e.g. PDE4 inhibitors, leukotriene antagonists, iNOS inhibitors, tryptase and elastase inhibitors, beta-2 integrin antagonists and adenosine 2a agonists.

The medicament is formulated as any suitable fluid formulation, particularly a solution (e.g. aqueous) formulation or a suspension formulation, optionally containing other pharmaceutically acceptable additive components. The formulation may contain a preservative, although the sealed system of the dispenser may negate the need for this.

The medicament formulation may incorporate two or more medicaments.

The dispenser herein is suitable for dispensing fluid medicament formulations for the treatment of inflammatory and/or allergic conditions of the nasal passages such as rhinitis e.g. seasonal and perennial rhinitis as well as other local inflammatory conditions such as asthma, COPD and dermatitis.

A suitable dosing regime would be for the patient to inhale slowly through the nose subsequent to the nasal cavity being cleared. During inhalation the formulation would be applied to one nostril while the other is manually compressed. This procedure would then be repeated for the other nostril. Typically, one or two inhalations per nostril would be administered by the above procedure up to three times each day, ideally once daily. Each dose, for example, may deliver 5 μg, 50 μg, 100 μg, 200 μg or 250 μg of active medicament. The precise dosage is either known or readily ascertainable by those skilled in the art.

It will be understood by the skilled reader in the art that the present invention is not limited to the embodiments herein described with reference to the FIGURES of drawings, but may be varied to adopt other guises within the scope of the appended claims. As an example, the dispenser of the invention need not be hand-held, nor hand-operable. Furthermore, the dispenser may be used to deliver any number of different fluid products, medicinal and non-medicinal, as outlined previously. Additionally, the dispenser may form an internal part of a device unit so that the dispenser delivers a metered volume of the fluid product to another internal part of the device unit. For instance, the unit may be a dispenser unit including the dispenser and the metered volume is delivered to conveying means in the dispenser unit which conveys the fluid product to an outlet orifice of the unit for discharge from the unit to the surrounding environment. The conveying means may be such as to change the state of the fluid, e.g. the conveying means may have a vibrating element, e.g. a mesh, which converts a metered volume of liquid to an aerosol or mist which is then directed out of the outlet orifice. The vibrating element could, for example, be a piezoelectric element or mesh.

Finally, for the avoidance of doubt, the inclusion of reference numerals in the claims is purely for illustration, and not meant to have a limiting effect on the scope of the claims.

1. A fluid dispenser for dispensing a metered volume of a fluid product having:
(a) a storage chamber for storing the fluid product in;
(b) a dispensing outlet through which the fluid product is dispensable from the dispenser;
(c) a metering chamber which is adapted to provide the metered volume of the fluid product for dispensing through the dispensing outlet by movement of the metering chamber between a contracted state and an expanded state, movement of the metering chamber from the contracted state to the expanded state placing the metering and storage chambers in fluid communication to enable the metering chamber to receive from the storage chamber an excess volume of the fluid product comprising the metered volume and a surplus volume;
(d) a bleed arrangement adapted to bleed the surplus volume of the fluid product from the metering chamber; and
(e) an actuating mechanism for actuating movement of the metering chamber between the expanded and contracted states;
wherein:
(f) the actuating mechanism has a manually-operable actuator member mounted on the dispenser for movement in a predetermined direction with respect to the dispenser; and
(g) the actuating mechanism is adapted to cause a cycle of movement of the metering chamber which takes in its expanded and contracted states in response to movement of the actuator member in the predetermined direction.

2. The dispenser in claim 1, wherein the metering chamber is defined by a boundary wall having a first section movably mounted in the dispenser to move the metering chamber between the expanded and contracted states.

3. The dispenser of claim 2, wherein the first section of the metering chamber boundary wall and the storage chamber are provided by a container unit which is movably mounted in the dispenser.

4. The dispenser of claim 1, wherein a transfer port is provided in the boundary wall through which the metering and storage chambers are places in fluid communication and the transfer port is selectively opened and closed when the metering chamber moves between its expanded and contracted states.

5. The dispenser in claim 4, wherein the transfer port is in the first section.

6. The dispenser of claim 4, wherein the transfer port is closed when the metering chamber is at an intermediate state between its expanded and contracted states.

7. The dispenser of claim 6, wherein the metering chamber has a volume corresponding to, or substantially corresponding to, the metered volume when at the intermediate state.

8. The dispenser of claim 6, wherein the transfer port is closed when the metering chamber moves between the intermediate and contracted states and open when the metering chamber moves between the intermediate and contracted states.

9. The dispenser of claim 1, wherein the boundary wall has a second section and the metering chamber is movable between its expanded and contracted states by movement of the first section in the dispenser relative to the second section.

10. The dispenser of claim 9, wherein the second section is stationary in the dispenser.

11. The dispenser of claim 4, wherein the boundary wall has a second section and the metering chamber is movable between its expanded and contracted states by movement of the first section in the dispenser relative to the second section, and wherein the second section is adapted in use to selectively open and close the transfer port.

12. The dispenser of claim 1, wherein an outlet port is provided in the metering chamber through which the metered volume of the fluid product is transferable from the metering chamber towards the dispensing outlet.

13. The dispenser of claim 12, wherein the boundary wall has a second section and the metering chamber is movable between its expanded and contracted states by movement of the first section in the dispenser relative to the second section, and wherein the outlet port is provided in the second section of the metering chamber boundary wall.

14. The dispenser of claim 3, wherein the container unit is adapted in use to operate as a pump mechanism for filling and emptying of the metering chamber.

15. The dispenser of claim 1, wherein movement of the metering chamber from its contracted state to its expanded state causes a pressure difference between the metering and storage chambers which results in the excess volume of the fluid product being drawn into the metering chamber.

16. The dispenser in claim 1, wherein movement of the metering chamber from its expanded state to its contracted state pumps the metered volume of the fluid product out of the metering chamber.

17. The dispenser in claim 1 in which the metering chamber is repeatedly movable between its different states thereby enabling the dispenser to repeatedly dispense a metered volume of the fluid product.

18. The dispenser of claim 1 further having a valve mechanism which is adapted in use to keep the dispensing outlet closed until the bleed arrangement bleeds the surplus volume of the fluid product from the metering chamber.

19. The dispenser of claim 18 in which the valve mechanism is adapted to open the dispensing outlet as the metering chamber moves to its contracted state and to re-close the dispensing outlet when the concentrated state is reached.

20. The dispenser of claim 12, further having a valve mechanism at the outlet port which is adapted to only allow the metered volume of the fluid product to be transferred to the dispensing outlet.

21. The dispenser of claim 20, wherein the valve mechanism is configured to close the outlet port except when the metering chamber moves to its contracted state after the bleed arrangement bleeds the surplus volume of the fluid product therefrom.

22. The dispenser of claim 18 in which the valve mechanism is a non-return valve mechanism.

23. The dispenser of claim 1 in which the dispensing outlet is in a nozzle of the dispenser.

24. The dispenser of claim 23, wherein the nozzle is configured as a mouthpiece or a nasal nozzle.

25. The dispenser of claim 1 in which the bleed arrangement is adapted in use to bleed the surplus volume of the fluid product in the metering chamber to the storage chamber.

26. The dispenser of claim 25, wherein a transfer port is provided in the boundary wall through which the metering and storage chambers are placed in fluid communication and the transfer port is selectively opened and closed when the metering chamber moves between its expanded and contracted states, and wherein the bleed arrangement is adapted in use to bleed the surplus volume of the fluid product to the storage chamber through the transfer port.

27-37. (canceled)

38. The dispenser of claim 3 in which the container unit is mounted for translational movement in the dispenser.

39. The dispenser of claim 1 having an axis along which the metering chamber moves to change its state.

40. The dispenser of claim 38, having an axis along which the metering chamber moves to change its state wherein the container unit translates along the axis.

41. The dispenser of claim 39 in which the storage and metering chambers are located on the axis.

42. The dispenser of claim 39, wherein an outlet port is provided in the metering chamber through which the metered volume of the fluid product is transferable from the metering chamber towards the dispensing outlet, and wherein the outlet port is located on the axis.

43. The dispenser of claim 39 in which the dispensing outlet is located on the axis.

44. The dispenser of claim 42 in which the dispensing outlet is located on the axis, and in which the outlet port and the dispensing outlet are at opposed ends of an axial channel of the dispenser.

45. The dispenser of claim 23 in which the storage chamber, metering chamber and nozzle are configured in-line.

46. The dispenser of claim 12 in which the storage chamber, metering chamber and outlet port are configured in-line.

47. The dispenser of claim 9, wherein the first section of the metering chamber boundary wall is mounted for sliding movement on the second section of the metering chamber boundary wall.

48. The dispenser of claim 47, wherein the first section of the metering chamber boundary wall is sealingly slidably mounted on the second section of the metering chamber boundary wall.

49. The dispenser of claim 39, wherein the first section of the metering chamber boundary wall is mounted for sliding movement on the second section of the metering chamber boundary wall, and wherein the first section of the metering chamber boundary wall presents at least a portion of an axially-oriented side of the metering chamber.

50. The dispenser of claim 49, wherein a transfer port is provided in the boundary wall through which the metering and storage chambers are placed in fluid communication and the transfer port is selectively opened and closed when the metering chamber moves between its expanded and contracted states, and wherein the transfer port is provided in the axially-oriented side of the metering chamber.

51. The dispenser of claim 2, wherein the first section of the metering chamber boundary wall presents a movable end wall of the metering chamber.

52. The dispenser of claim 2 in which the first section of the metering chamber boundary wall has a generally U-shape.

53. The dispenser of claim 49, wherein the first section of the metering chamber boundary wall presents a movable end wall of the metering chamber in which the first section of the metering chamber boundary wall has a generally U-shape, and wherein the end wall of the metering chamber is presented by the base of the U-shape and the side of the metering chamber is presented by the limbs of the U-shape.

54. The dispenser of claim 49, wherein the boundary wall has a second section and the metering chamber is movable between its expanded and contracted states by movement of the first section in the dispenser relative to the second section, and wherein the second section of the metering chamber boundary wall is presented by a structure having an axially-oriented surface on which the side of the metering chamber is slidably mounted.

55. The dispenser of claim 54, wherein the axially-oriented surface of the structure is an outer surface.

56. The dispenser of claim 9, wherein the second section of the metering chamber boundary wall presents an end wall of the metering chamber.

57. The dispenser of claim 9, wherein the second section of the metering chamber boundary wall is presented by a generally U-shape structure.

58. The dispenser of claim 54 wherein the second section of the metering chamber boundary wall presents an end wall of the metering chamber, and wherein the second section of the metering chamber boundary wall is presented by a generally U-shape structure in which the base of the U-shape structure presents the end wall of the metering chamber and the limbs of the U-shape structure present the axially-oriented surface.

59. The dispenser of claim 3 in which the first section of the metering chamber boundary wall is formed by a female depression in an outer surface of the container unit.

60. The dispenser of claim 59 wherein the boundary wall has a second section and the metering chamber is movable between its expanded and contracted states by movement of the first section in the dispenser relative to the second section in which the second section of the metering chamber boundary wall is formed as a male projection which is inserted into the female depression.

61. The dispenser of claim 59 in which the depression extends into the storage chamber.

62. The dispenser of claim 61 in which the storage chamber surrounds the depression.

63. The dispenser of claim 1 in which at least a portion of the storage chamber surrounds the metering chamber.

64. The dispenser of claim 63 in which the at least a portion of the storage chamber in concentrically arranged with the metering chamber.

65. The dispenser of claim 1 in which the metering chamber has zero volume, or substantially zero volume, when in its contracted state.

66. The dispenser of claim 65, wherein the boundary wall has a second section and the metering chamber is movable between its expanded and contracted states by movement of the first section in the dispenser relative to the second section, and wherein the first and second sections of the metering chamber boundary wall abut in the contracted state.

67. The dispenser of claim 66, wherein the first and second sections of the metering chamber boundary wall are of complementary shape.

68. The dispenser of claim 66 in which the first and second sections nest in the contracted state.

69. The dispenser of claim 13 in which the first section of the metering chamber boundary wall closes off the outlet port in the contracted state of the metering chamber.

70. The dispenser of claim 1 which is hand-held.

71. The dispenser of claim 3 in which the actuator member is operatively coupled to the container unit to move the container unit such that the metering chamber completes the cycle between its different states.

72. The dispenser of claim 1 in which the predetermined direction is inward with respect to the dispenser.

73. The dispenser of claim 72 in which the actuator member is biased in an outward direction.

74. The dispenser of claim 1 in which the actuator member is a trigger member.

75. The dispenser of claim 1 in which the actuator member is pivotably mounted on the dispenser.

76. The dispenser of claim 1 in which the dispensing outlet is located at an upper end of the dispenser and the actuator member is mounted on a side of the dispenser.

77. The dispenser of claim 75, in which the dispensing outlet is located at an upper end of the dispenser and the actuator member is mounted on a side of the dispenser wherein the actuator member has a pivot point at a lower end thereof.

78. (canceled)

79. The dispenser of claim 78, wherein the first section of the metering chamber boundary wall and the storage chamber are provided by a container unit which is movably mounted in the dispenser, and wherein in the rest condition the container unit is disposed in a rest position in the dispenser and the actuating mechanism is adapted to move the container unit through a cycle which commences, and ends, at the rest position and passes through a priming position, in which the metering chamber is in its expanded state, upon actuation of the actuating mechanism.

80. The dispenser of claim 79 in which the actuating mechanism biases the container unit to the rest position.

81-84. (canceled)

85. The dispenser of claim 1 in which the bleed arrangement is adapted such that the surplus volume of the fluid product is caused to bleed from the metering chamber by movement of the metering chamber from the expanded state towards the contracted state

86. The dispenser of claim 3, wherein the actuating mechanism has a drive structure which is adapted in use to drivably move the container unit in a first direction responsive to movement of the actuator member in the predetermined direction thereby moving the metering chamber towards one of its different states.

87. The dispenser of claim 86, wherein movement of the container unit in the first direction causes the metering chamber to move from its contracted state to it expanded state.

88. The dispenser of claim 86 in which the first direction is generally transverse to the predetermined direction.

89. The dispenser of claim 86 in which the drive structure is coupled to the actuator member so as to be moved thereby.

90. The dispenser of claim 89 in which the drive structure is pivotably coupled to the actuator member.

91. The dispenser of claim 86, wherein the drive structure is a pusher structure for pushing the container unit in the first direction.

92. The dispenser of claim 86, wherein the drive structure is a cam structure for camming the container unit in the first direction.

93. The dispenser of claim 1, wherein the actuating mechanism is adapted in use to move the metering chamber from its contracted state to its expanded state in a first phase of movement of the actuator member in the predetermined direction and wherein the metering chamber returns to its contracted state in a second phase of movement of the actuator member in the predetermined direction which follows the first phase.

94. The dispenser of claim 93, wherein the actuating mechanism has a biasing structure which biases the metering chamber to its contracted state, wherein in the first phase the metering chamber expands against the biasing force of the biasing structure and further wherein in the second phase the biasing force is allowed to bias the metering chamber back to the contracted state.

95. The dispenser of claim 94, wherein the fluid is a liquid and the biasing force of the biasing structure is sufficient to cause the metered volume of the liquid to be dispensed from the dispensing outlet as an atomised spray by the movement of the metering chamber back to its contracted state in the second phase of movement of the actuator member in the predetermined direction.

96. The dispenser of claim 94 when appended to claim 3, wherein the biasing structure acts on the container unit.

97. The dispenser of claim 94, wherein the actuating mechanism has a drive structure which is adapted in use to drivably move the container unit in a first direction responsive to movement of the actuator member in the predetermined direction thereby moving the metering chamber towards one of its different state, and wherein the drive structure disengages from the container unit in the second phase to enable the biasing structure to return the metering chamber to its contracted state.

98-103. (canceled)

104. A dispenser unit having a dispenser according to claim 1 in which the dispensing outlet is a dispensing outlet of the unit through which the metered volume of the fluid product is, in use, dispensed to the external environment.

105-110. (canceled)
